(19) United States
(12) Patent Application Publication
(10) Pub. No.: US 2003/0040485 A1
(43) Pub. Date: Feb. 27, 2003

(54) alpha/beta-adrenoceptors blockers and angiotensin converting enzyme inhibitors derived from hydroxyphenyl carboxylic acid and alcohol

(76) Inventor: Ing-Jun Chen, Kaohsiung (TW)

Correspondence Address: DENNISON, SCHULTZ & DOUGHERTY, 1745 JEFFERSON DAVIS HIGHWAY, ARLINGTON, VA, 22202, US

(21) Appl. No.: 10/136,494

(22) Filed: May 2, 2002

(30) Foreign Application Priority Data
May 4, 2001 (TW) 090110991

(51) Int. Cl. A61K 38/05; A61K 31/137; C07K 5/062

(52) U.S. Cl. 514/19; 514/538; 514/652; 548/530; 560/39; 564/349

(57) ABSTRACT

The invention disclosed some derivative chemically with Hydroxyphenyl carboxylic acid and Alcohol based phenoxypropanolamine and associated alanyl-proline peptide derivatives. The compounds shown as formula I,

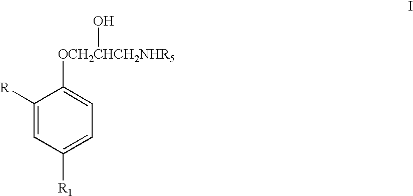

whether

R is a number selected from the group of hydrogen, unsaturated 2-6 straight chain carbon atoms, saturated 1-6 straight chain carbon atoms, unsaturated 2-6 straight chain of alkoxyl group, saturated 1-6 straight chain of alkoxyl group, halogen, and —NO₂;

R₁ is selected from the groups of —R₂OH, —R₂OR₃, —R₂COCOOR₄, —R₂COOR4 and R₂CH(COR₄)-alanylproline, R₂CH(COOR₄)-alanylproline;

R₂ is selected from the groups of unsaturated 2-6 straight chain carbon atoms, saturated 1-6 straight chain carbon atoms;

R₃ is selected from the ester groups consisting of proline and alanylproline;

R₄ is selected from the groups of hydrogen, unsaturated 2-6 straight chain carbon atoms, saturated 1-6 straight chain carbon atoms;

R₅ is selected from the groups of hydrogen, unsaturated 2-6 straight chain carbon atoms, saturated 1-6 straight chain carbon atoms, and

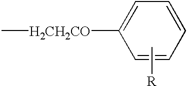

R may be on the meta, ortho, or para position with ethoxyl group on the bezene ring.

Through in vivo experiment to prove those compounds have new generation α/β-adrenoceptor antagonist with vasorelaxant activity or angiotensin converting enzyme inhibitory activities.

### BACKGROUND OF THE INVENTION

1. Field of the Invention

This invention related to compounds of Hydroxyphenyl carboxylic acid substituted derivatives, which upon laboratory testing on animals, have proven that they pharmacologically possess Angiotensin Converting Enzyme Inhibitor (ACEI) activity.

2. Description of the Prior Art

In recent years, some β-adrenoceptors blockers such as eugenodilol, ferulidilol, vanidipinedilol, labedipinedilol-A, carvedilol, that have better function then others vasorelaxant, have been reported by Huang Y C. et al. (J Cardiovasc Pharmacology, vol.34, pp.10˜20, 1999. and Drug Development Research, vol.47, pp.77˜89, 1999). Those information inspired to devise some high potency β-adrenoceptors blockers, α/β-adrenoceptor blockers, and Angiotensin Converting Enzyme Inhibitivity (ACEI) compounds.

In recent years, inventor have studied various guaiacoxy and vanilloid-based derivatives that have potent β-adrenoceptors blocking activity. Those β-adrenoceptor blockers have chemically with 3-methoxy-4-hydroxy benzyl group found in natural compounds as roughly separated into two types. One type is nature derivatives from xanthone, reported by Chen I. J. et al. (Gen Pharmacol, vol.24, pp.1425-1433, 1993). Second type is synthesized compounds that started from capsaicin reported by Chen I. J. et al. (Journal of Medicinal Chemistry vol.37, pp. 938˜943, 1994). From vanillin and dehydrozingerone was reported by Sheu M M et al. (Pharmacology, vol.54, pp.211˜224, 1997). From eugenol reported by Wu B. N. et al. (Asia Pacific Journal of Pharmacology, vol.11, pp.5˜12, 1996), and Chen S J et al. (Drug Development Research, vol.40, pp.239˜250,1997). Or from guaiacoxypropanolamine reported by Chen I. J. et al. (British Journal of Pharmacology, vol.119, pp.7˜14, 1996)

### SUMMARY OF THE INVENTION

This invention covers new compounds of phenoxypropanolamine and peptide substituted derivatives, which on laboratory testing in animals, have proven that they pharmacologically possess β-adrenoceptors, α/β-adrenoceptors, binding affinity, and Angiotensin Converting Enzyme Inhibitivity (ACEI).

This invention also covers some phenoxypropanolamine and peptide substituted derivatives, testing in animals, have proven they possess β-adrenoceptors, α/β-adrenoceptors, binding affinity, and Angiotensin Converting Enzyme Inhibitivity (ACEI). Anyone who is familiar with the said technique that described by way of example with reference to the accompanying Tables and Figures is able to understand the purpose and excellence of the invention.

### BRIEF DESCRIPTION OF THE DRAWINGS

The invention will now be described by way of example with reference to the accompanying Tables and Figures in which.

Table 1 illustrates physicochemical Data of compounds C, H, N were analyzed: the values are at ±0.4% of the theoretical values.

Table 2 illustrates decrease of blood pressure affect of compounds

Table 3 illustrates pA₂ and β₁/β₂-Selectivity Values from In Vitro Experiments

Table 4 Inhibitory activity of angiotensin converting enzyme (ACE) by various ACE inhibitors

FIG. 1 illustrates the synthetic method of the compounds 4

FIG. 2 illustrates the synthetic method of the compounds 6

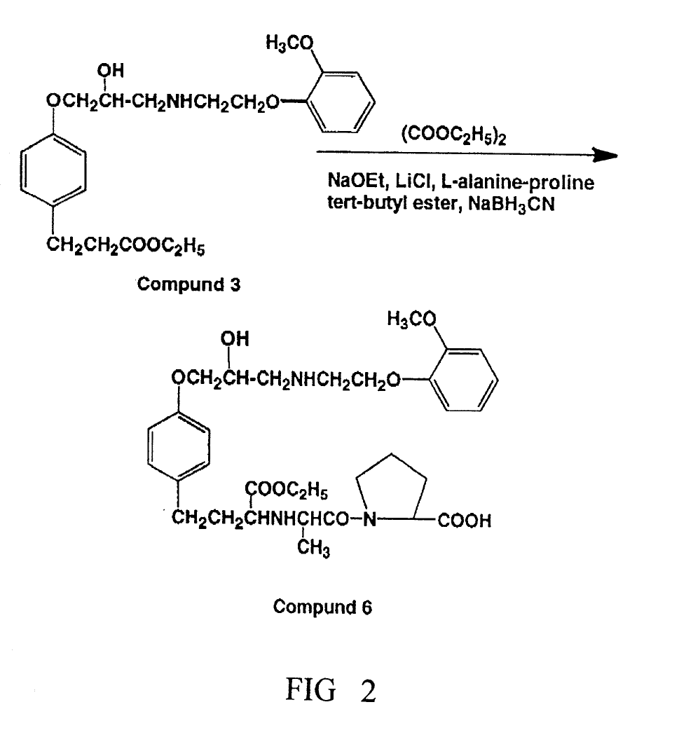

### DETAILED DESCRIPTION OF THE INVENTION

The invention covered phenoxypropanolamine and peptide substituted derivative of formula I, which upon laboratory testing on animals, had proven that they pharmacologically possess β-adrenoceptors, α/β-adrenoceptors, binding affinity, and Angiotensin Converting Enzyme Inhibitivity (ACEI).

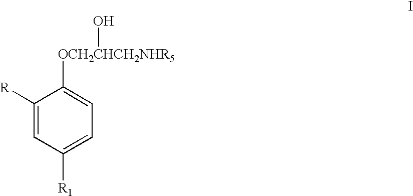

Whether R is selected from groups of hydrogen, unsaturated 2-6 straight chain carbon atoms, saturated 1-6 straight chain carbon atoms, unsaturated 2-6 straight chain of alkoxyl group, saturated 1-6 straight chain of alkoxyl group, halogen, and —NO₂;

R₁ is selected from the groups of —R₂OH, —R₂OR₃, —R₂COCOOR₄, —R₂COOR₄, R₂CH(COR₄)-alanylproline, and R₂CH(COOR4)-alanylproline;

R₂ is selected from the groups of unsaturated 2-6 straight chain carbon atoms, saturated 1-6 straight chain carbon atoms;

R₃ is selected from the ester groups of peptides of ACEI (Angiotensin Converting Enzyme Inhibitor) terminal structure;

R₄ is selected from the groups of hydrogen, unsaturated 2-6 straight chain carbon atoms, saturated 1-6 straight chain carbon atoms;

R₅ is selected from the groups of hydrogen, unsaturated 2-6 straight chain carbon atoms, saturated 1-6 straight chain carbon atoms, and

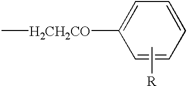

R may be on the meta, ortho, or para position with ethoxyl group on the bezene ring.

The said R₃ is selected from the ester groups of peptides with ACEI (Angiotensin Converting Enzyme Inhibitor) terminal structure. Whether peptide in special of proline and alanylproline, the ACE inhibitors are captopril, enalapril, and lisinopril. The preparation of esters were by esterification of alcohol with carbonium group of described amino acids.

The prepared method of vanilloid-based derivatives is under alkaline solution to react starling material with epichlorohydrin. Whether intermediate epoxy group compound then react with coxyethylamine or tert-butylamine.

The characterics of invention is that they have alanylproline group on these novel compounds to enhance the effects of ACEI (Angiotensin Converting Enzyme Inhibitor), which were chemically lack of α/62 -adrenoceptors blocking associated moiety, such as phenoxypropanolamine and guaiacoxypropanolamine. These novel compounds of invention possesse better antihypertensive benefits for therapeutics of some associated disease, since they have two effects that derived from ACEI and α/β-adrenoceptors blockers.

Preparation of the type I vanilloid-based derivatives have some methods, usually under alkaline solution to react starting material with epichlorohydrin. Whether intermediate compounds with epoxy group then reacted with guaiacoxyethylamine or tert-butylamine, respectively.

Added 4-hydroxylphenylpropionic acid and ethanol into reaction bottle, reaction was carried out with NaOH and epichlorohydrin. Till complete the reaction, then removed the solvent, purified by chromatography, to obtain the pure white crystal compound 4-epoxylphenylpropionic acid ethyl ester. For continue refluxed 4-epoxylphenylpropionic acid ethyl ester with guaiacoxyethylamine in anhydrous alcohol. Then removed the solvent under reduced pressure, and purified by chromatography, eluated with equal ratio of n-hexane and ether, then recrystalized to obtain the pure compound 3 (Propiodilol).

Following the same process and replacing 4-hydroxylphenylpropionic acid with vanillyl alcohol, compound 4 (Vanydilol) were obtained.

Dissolved 4-epoxylphenylpropionic acid ethyl ester and tert-butylamine in anhydrous alcohole, continued to reflux till to complete the action. Then removed the solvent, purified by chromatography, to obtain the pure white crystal compound then recrystalized with ethanol and ether to obtain the pure compound 1 (Propionolol). Follow the same process and replacing 4-epoxylphenylpropionic acid ethyl ester with 4-epoxyphenylmethyl alcohol, compound 2 (Vanynolol) were obtained.

Mixed compound 3 (Propiodilol), NaOEt, and (COOEt)₂, and heated to 60-70° C., cooled and distilled water was added into and neutralized with conc. HCl. Divided the ethyl acetate layer at separate funnel, add DMSO into aqua layer, then continue refluxed after add aqua solution mixture of LiCl. Cooled the mixture and extract with ethyl acetate, to obtain the hazel aqua solution. Then purified by chromatography, eluated with equal ratio of Methanol and ethyl acetate, recrystalized with methanol, to obtain the pure white compound.

Dissolved the pure white compound in ethanol-L-alanine-proline tert-butyl ester solution and continued to stirr over night after added with ethanol-sodium cyanoborohydride. Cooled the mixture and extract with ethyl acetate, dried with anhydrous NaSO₄, the solvent was removed under reduced pressure. Dissolved residuer with n-hexane, continued to reflux after added with CH₃COOH. Then removed the solvent and recrystalized with Methanol, to obtain the compound 6 (Labetapril).

On the other hands, compound 1 (Propionolol) was heated with NaOEt and (COOEt)₂, then cooled and distilled water was added and neutralized with conc. HCl. Divided the ethyl acetate layer at separate funnel, added with DMSO into aqua layer, then continued to reflux after added with aqua solution mixture of LiCl. Cooled the mixture and extract with ethyl acetate, to obtain the hazel aqua solution. Then purified by chromatography, eluated with equal ratio of Methanol and ethyl acetate, recrystalized with Methanol, to obtain the pure white compound.

Dissolved the pure white compound in ethanol-L-alanine-proline tert-butyl ester solution and continue stirre over night after added ethanol-sodium cyanoborohydride. Cooled the mixture and extract with ethyl acetate, dried with anhydrous NaSO₄, the solvent was removed under reduced pressure. Dissolved residuer with n-hexane, continue refluxed under after added CH₃COOH, Proline benzyl ester-DEPC solution and Et₃N-DMF solution. Wash with mixture solution of ethyl acetate, 10% H₃PO₄, 1N NaOH, and H₂O, when the reaction completed. Then removed the ethyl acetate layer to obtain the white solid.

Dissolved the white solid with CH₃COOH, continued to reflux for 1 hour, then added with diethyl phosphorocyanidate (DEPC). The concentrate was purified by chromatography, eluated with n-hexane and ethyl acetate, to obtain the compound. recrystalized with methanol, to obtain the compound 6 (Labetapril).

The diethyl phosphorocyanidate (DEPC) in reaction was used as condensation agent.

After purification and crystallization, the products are individually tested for their physio-chemical properties, including elementary analysis MS, IR, ¹H-NMR (CDCl₃), and UV. The results are shown in Table 1. Appropriate experimental models are used to evaluate their pharmacological activities, thus ascertaining the compounds activity. Table 2 shown maximal decrease of blood pressure affect of compounds. Table 3 shown pA₂ and β₁/β₂-Selectivity Values. Table 4 shown Angiotensin Converting Enzyme Inhibitivity (ACEI) activity.

The compositions of this invention will include various excipients; carriers or diluents and pharmaceutically salts, in accordance with the necessity to form compositions with therapeutic efficacy. These pharmaceutical preparations may be in solid form for oral and rectal administration; liquid form or non-intestinal injection form; or ointment form for direct application on affected part. Such solid forms are manufactured according to common pharmaceutical preparation methods, which will include disintegrant like starch; sodium carboxymethylcellulose, adhesive like ethanol; glycerine, or magnesium stearic acid; lactose to obtain pharmaceutical preparation like tablets or filled into capsules of suppositories. Solution which include a compound of this ingredient could use buffers of phosphoric nature to adjust the pH to suitable level, before adding the adjutant; emulsifiers to produce injection dose or other liquid preparation. In the present invention a compound or a pharmaceutical composition could be manufactured by mixing synthetic acid salts with various fundamental preparations to form ointments according to known pharmaceutical manufacturing methods. Pharmaceutical compositions manufactured according to this invention could be used on mammals to produce the efficacy of the main ingredient. General dosage could be adjusted according to the degree of symptoms, and normally a person will require 50 to 300 mg each time, three times per day.

The invented compounds upon laboratory testing in animals, proven that they possessed α/β-adrenoceptor binding affinity, and Angiotensin Converting Enzyme Inhibitito (ACEI) activity. Since the structure have two basical moiety group; one is aryloxypropanol which possesses β-adrenoceptors-blocking activity. Another is guaiacoxyethylamine, which possessed α-adrenoceptors-blocking activity.

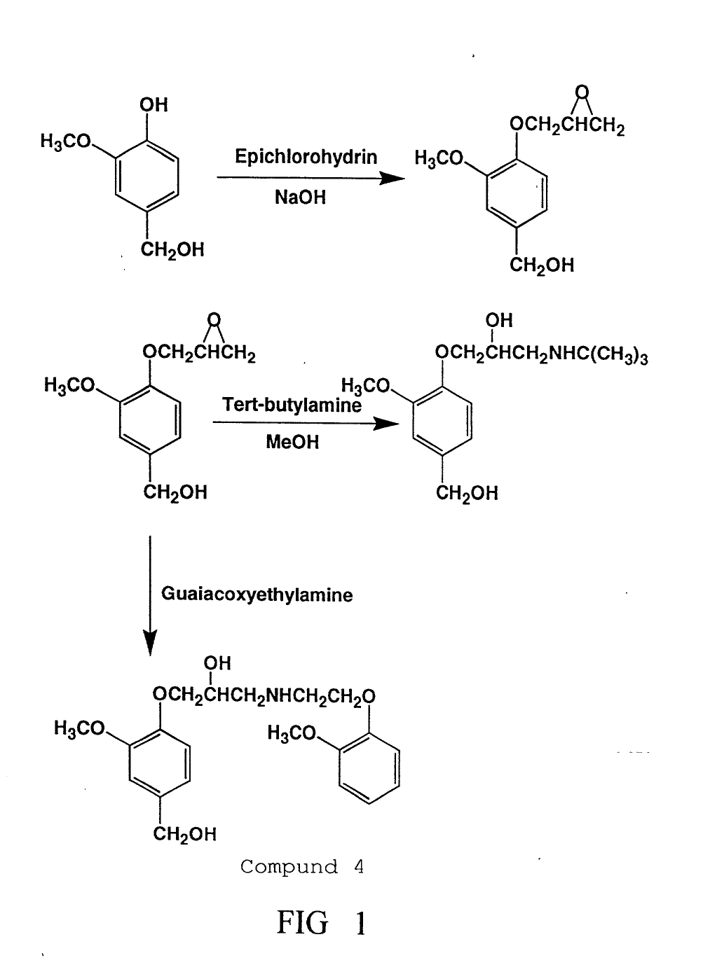

Special derivated compounds of 4-hydroxy-phenylpropionic acid and vanillin alcohol such as compound 1, and compound 3 are shown on FIG. 1, they have various β-adrenoceptors-blocking activities. The compound 2 and compound 4 has α/β-blocker activity. Compound 5-8 not only possesses α/β-blocker activity, but also has Angiotensin Converting Enzyme Inhibitor activity.

The compounds of invention upon in vivo and in vitro testing, proven that have pharmacologically possess antihypertension, α/β-adrenoceptor binding affinity, Intrinsic Sympathomimetic Activity(ISA), vascular smooth muscle relaxant activities, and Angiotensin Converting Enzyme Inhibitivity (ACEI).

### 1. Heart Rate and Blood Pressure in Living Rat

Normotensive Male Wistar rats (NTRs), weighing 200˜300 g were abdominal anaesthetized with pentobarbital sodium. Tracheal was cannulated to maintain normal respiration. Polyethylene tube was inserted into the left femoral vein to facilitate drug administration. A 3-way stopcock was used, with one end comnected to a syringe for drug injection, while the other end was connected to the syringe filled with physiological saline. The latter was used to prevent residual drugs in the polyethylene tube after injection, which would affect experimental accuracy.

The right femoral artery was also inserted with polyethylene, and a 3-way stopcock was used tool, where one end was connected to heparin solution to prevent embolism. The other end was comnected to a Disposable Diaphragm Dome, TA1019, and linked to a pressure transducer (Model P10EZ; Spectramed, Oxnard, Calif., USA). Through an amplifier, a recorder recorded the overall and average arterial pressure; heart rate to evaluate the effect of drug on blood pressure and heart rate. Different concentrations 0.1, 1.0, 2.0, 3.0 mg/kg of test compounds were given to the rats via femoral vein and the differences in the heart rate and blood pressure were compared. Table 2 shown maximal decrease of blood pressure affect of compounds after given 2.0 mg/kg test compounds to the rats.

### 2. Examination of the Effect on α/β-adrenoceptors-blockers

To determine the heart rate as same as starting, the Normotensive Male Wistar rats (NTRs) was administrated 5 mg/kg ganglion blocker mecamylamine. Tachycardia was induced after administrated 0.5 μg/kg L-isoproterenol into thigh vein. Then following injected single dose of compound 1-4, after 10 minute, administrated L-isoproterenol to obtain contrast response of control group.

At other group pre-administrated 5 mg/kg reserpine into vein, after 24 hrs, 10 μg/kg (−)L-phenylephrine have inject, following in proper order administrated 0.5 mg/kg isoeugenodilol (15) or 1.0 mg/kg propranolol at 15 minute, then re-injected (−)L-phenylephrine after 10 minute.

### 3. Evaluation of Relaxation of Blood Vein

After sacrificing Wistar rat of weight between 300˜450 gm, the thoracic aorta was immediately removed and placed in cold Kreb's solution. The fatty connecting tissue surrounding the vessel wall was removed and the thoracic aorta was cut into rings of length 5 mm. The two ends of each ring was pieced and fixed with “Z” shaped platinum wires. Then the thoracic aorta was suspended in 10 ml of tissue bath, aerated with air mixture (95% O₂+5% CO₂) and maintained at 37° C., where one end was fixed at the bottom of tissue trough, the other end connected to force transducer to record the long contraction via recorder. The sample was given 1 gm of tension and equilibrated for 60 minutes before the following experiments were carried out.

The specimen is given one gram of tension, and after balancing for 60 minutes, 10 μM of phenylephrine is prescribed to test the vivacity of the main chest artery. Upon balancing and the phenylephrine rinsed off, 10 μM of phenylephrine is used again to achieve the maximum contraction. And upon balancing, different concentration of synthetic testing compounds are used, 10⁻⁵ M, 10⁻⁶ M, or 10⁻⁷M, to observe the suppressing effects resulted from different concentrations of synthetic testing compounds.

### 4. In vitro Evaluation of Vasodilating Activity

Vascular α-adrenoceptor blocking activity was evaluated in the Wistar rat aorta preparation. Isolated rat aorta preparations were mounted in organ bath and suspended in Krebs solution at 37° C. under a resting tension of 3×10⁻⁶ M norepinephrine or 75 mM KCl was added to the bath medium after a control concentration-response curve to norepinephrine had been obtained. The tissue was exposed to test compounds for 30 min before rechallenging with norepinephrine. Inhibitions were expressed as percentages of the maximum contraction obtained by the challenge of the tissues with norepinephrine. The 70 mM NaCl on 75 mM KCl solution may to replace with equimolar KCl, if necessary.

### 4-1. α-Adrenoceptor Antagonism

The methods published by Huang Y C. et. al. in J Cardiovasc Pharmacol.34, pp.10-20 (1999) and Drug Devel Res. 47, 77-89 (1999). Through referenced and modified method to evaluate, test compounds whether have α₁-adrenergic and sertoninergic effect.

Isolated Wistar rat aorta preparations were mounted in organ bath and suspended in Krebs solution (NaCl, 118 mM, KCl, 4.8 mM; CaCl,2.5 mM; MgSO₄,1.2 mM; K₂PO₄,1.2 Mm; CaHCO₃,24 Mm; glucose, 11 mM) at 37° C. under 1 g resting tension.

Upon balancing for 60 minutes, 10⁻¹⁰-10⁻⁴ M norepinephrine is administered directly from low concentration to high using 0.5 log unit to gradually increase until the maximum reaction is reached, and this act as the control unit. Then different concentrations of the synthetic testing compounds, 10⁻⁷ M, 10⁻⁶ M, or 10⁻⁵M, are used, and upon 30 minutes of reaction, norepinephrine is used again to complete the cumulative dose. The EC₅₀ value of blocking serotonin may be obtained from observation of the suppressing effects caused by different concentrations of the synthetic testing compound.

### 4-2. β₁- and β₂-Adrenoceptor Antagonism

The spontaneously-beating right atria was clipped on both end by heart shaped clips, where one end was fixed at the bottom of 10 ml of tissue bath made of physiological saline solution, and temperature maintained at 37° C. The other end of the atria was connected to a force transducer, and isometric contractions and beating rate of the right atria were recorded by COULBOURN AT-High-Speed VideoFigure. After the samples were given 250 mg of contractions and reach equilibrium the following experiments were carried out:

When the spontaneously beating rate of right atria reached a certain stability, cumulative administration of L-isoproterenol from 1×10⁻¹⁰˜3×10⁻¹⁰ M caused the heart rate to increase continuously, and a cumulative dose-response curve was obtained. Then the L-isoproterenol was thoroughly washed off with Kreb's solution to recover the right atria's heart rate stability. After the equilibrum was reached again for at least 60 minutes, different concentrations (10⁻⁷, 10⁻⁶, 10⁻⁵ M) of compounds were added. 30 minutes later, cumulative administration of L-isoproterenol from 1×10⁻¹⁰˜3×10⁻¹⁰ M were carried out again, and another new cumulative dose-response curve was obtained. Administration of L-isoproterenol started from concentration 1×10⁻¹⁰ M, and the concentration was raised 0.5 log each time for a total of six times. Cumulative administration interval was when the previous concentration reached its greatest effect, the next concentration would be immediately given. The time interval was approximately 3˜5 minutes, and the EC₅₀ value could be obtained. From Schild plots, the pA₂ of compounds could be found. In other groups of rats, after separate administration of test compounds, their pA₂ values were obtained.

### 4-3. Calculation of pA₂ Value

According to the method mentioned by Arunlakshana, O. et al. in Br. J. Pharmacol. 14, pp. 48-57 (1959), which used the logarithm values of compound concentration testings as the x-coordinates, and the logarithm values of blocking agent of similar effect and (dose ratio)⁻¹ as the y-coordinates, the data obtained were plotted into Figures and the slope of regression found. From x-coordinates of the line of regression, the intercept value was found, which is the pA₂ value of the compound under testing. The equation is as follows:

pA₂=−Log KB Log(DRADJ−1)=n log[B]−LogKB

$$DRADJ ( dose ratio adjusted ) = \frac{DR ( dose ratio )}{CF ( correction factor )}$$

[B]=Test compound concentration in moles

KB: equilibrium dissociation constant

n: value of slope

DR: test EC₅₀ divided by control EC₅₀

CF: EC₅₀ of second or third control groups divided by EC₅₀ of first control group

### 5. Adrenoceptor-Binding Assay

### 5-1. Adrenoceptor-Binding Assay for Evaluated the Binding Effect of β-adrenoceptor Antagonist and [³H]CGP-12177

According to the method mentioned by Huang Y C.(J Cardiovasc Pharmacol.vol.34, pp.10-20, 1999 and Drug Devel Res. Vol.47, pp.77-89, 1999), the Wistar rat were sacrificed, the ventricle, lungs and brain tissue were immediately separated. The Bradford et. al. (1976) instrument Protein content method were adopted with modifications.

[³H]CGP-12177 and ventricle or lung membranes (200-300 μg) were incubated for 60 min at 25° C., with and without the addition of 10 μM propranolol. in 75 mM Tris-HCl buffer with 25 mM MgCl₂, to make a final volume of 250 μl.

In competitive-binding experiments, the competing agent was added directly to the incubation mixture. The incubation was terminated by addition of 1 ml of ice-cold assay buffer followed by immediate filtration through Whatman GF/C glass fiber filters supported on a 12 port filter manifold (Millipore). The filters were immediately washed 3 times with 5 ml of ice-cold assay buffer and dried in an oven at 80° C. for 2 h before adding 5 ml of Triton/toluene-based scintillation fluid. Membrane-bound [³H]CGP-12177 trapped in the filters was counted in a Beckman LS 6500 scintillation system (Fullerton. Calif. U.S.A.) with an efficiency of 45%. In each experiment, nonspecifically bound [³H]CGP-12177 was determined by incubating membrane protein and [³H]CGP-12177 with 10 μM propranolol. Specific binding was thus obtained by deducting this value from the total binding of [³H]CGP-12177 for each sample.

Calcium Ion Receptor Binding

The total binding value−unparticular-binding value=particular-binding value

The unparticular-binding [³H]CGP-12177 value determine by [³H]CGP-12177 and membranes protein cultivat with 10 μM propranolol.

### 5-2. Specific α-Adrenoceptor Antagonist

According to the method of β-adrenoceptor antagonist and [³H]CGP-12177.[³H]prazosin and ventricle or lung membranes (250-300 g) were incubated for 60 min at 37° C., with and without the addition of 0.1 mM phentolamine in 50 mM Tris-HCl buffer with 10 mM MgCl₂, to make a final volume of 500 μl.

After competitive agent directly add into culture solution reaction, 1 ml of cold Tris buffer was added to terminate the binding reaction. Then Millipore filtration manifold (Millipore, Bedford, Mass., USA) and Whatman GF/C glass fiber were used for rapid press filtration, and 5 ml of cold Tris buffer was used to rinse the filtrate three times. After the filter paper with the filtrate was dried in a 80° C. oven for 2 hours, 4 ml of Triton-toluene based scintillation fluid was added, and Beckman LS6500 rackbeta liquid scintillation counter (Fullerton, Calif., U.S.A.) was used to determine the strength of radioactivity, when [³H]prazosin to covered filtrate.

Membrane-bound [³H]prazosin trapped in the filters was counted in a Beckman LS 6500 scintillation system (Fullerton. Calif. U.S.A.) with an efficiency of 45%. In each experiment, nonspecifically bound [³H]prazosin was determined by incubating membrane protein and [³H]prazosin with 0.1 mM phentolamine. Specific binding was thus obtained by deducting this value from the total binding of [³H]prazosin for each sample.

Calcium Ion Receptor Binding

The total binding value−unparticular-binding value=particular-binding value

The unparticular-binding [³H]prazosin value determine by [³H]prazosin and membranes protein cultivat with 0.1 mM phentolamine.

### 6. Determine ACEI(Angiotensin Converting Enzyme Inhibitor) Activity

ACE value in serum was determined by Hycor kit, rat serum were cultured in 37° C. of [³H]-hippuryl-glycyl-glycine for 60 minutes. While [³H]-hippuryl-glycyl-glycine contains three peptide. [³H] hippuric acid was obtained when extracted of culture solution with ethyl acetate, the content be determined by spectrophotometer. The content in serum sample was as the control group after it was added with 10⁻⁸M, 10⁻⁷M, 10⁻⁶M compounds, then dissolved in PBS.

### 6-1. ACE Activity Value on Tissue

Centrifuged 250-300 mg lung tissue, 3 ml extracted liquid (10 mmol/L HEPES, 125 mmol/L NaCl, pH7.4) under 12000 g for 20 minutes at 4° C. by Teflon-glass homogenizer, the cell membrane was obtained. The 3 ml homogenized liquid was added into twice, before and after centrifuged. Then the Lowry's method was adopted to determine the protein content in the membrane. Mixing 15 minute of liquid and 30 mmol/L Hip-His-Leu solution at 37° C. Fluorometric was used to determine the Hippurate content. test compounds (10⁻⁸M, 10⁻⁷M, 10⁻⁶M) dissolved in PBS normal saline and used as control group. Then ACE activity inhibition rate was determined.

### EXAMPLE 1

### Synthesis of 4-epoxylphenylpropionic Acid Ethyl Ester

15 g 4-hydroxylphenylpropionic acid pour into reaction bottle and dissolved in 900 mL ethanol, same mole amount of NaOH was added and carried out the reaction for 1 hour under 70° C. 5 fold moles epichlorohydrin was added continue refluxed for 2 hrs, under similar condition to complete the reaction. Then removed the solvent under reduced pressure, and pour concentrator into silica gel column, purified by chromatography, eluated with equal ratio of n-hexane and ethyl acetate, to obtain the pure white crystal compound.

### EXAMPLE 2

### Synthesis of 4-epoxylbenzylalcohol

15 g vanillyl alcohol was poured into reaction bottle and dissolved in 900 mL ethanol, same mole amount of NaOH was added and the reaction was carried our for I hour under 70° C. 5 fold moles epichlorohydrin was added continue refluxed for 2 hrs, under similar condition to complete the reaction. Then the solvent was removed under reduced pressure, the concentrator was pour into silica gel column, purified by chromatography, eluated with equal ratio of n-hexane and ethyl acetate, to obtain the pure white crystal compound.

### EXAMPLE 3

### Synthesis of Guaiacoxyethylamine

(1) Prepared Guaiacoxyethylbromide

Mixed 0.2 mole (22.4 ml) 2-methoxyphenol and 0.4 mole (34.6 ml) ethylene dibromide then heated to 100° C., severe stirring and added 125 mL 1.6N NaOH solution before 30 minutes continue refluxed and stirring till pH 7. Cooled the mixture and extract with CH₃Cl, wash the organical layer in turn with 2N NaOH, saturated NaCl solution and MgSO₄. Then the solvent was removed under reduced pressure, the concentrator was pour into silica gel column, purified by chromatography, eluated with equal ratio of n-hexane and ethyl acetate, to obtain the pure white crystal compound.

(2) Prepared Guaiacoxyethylphthalimide

0.156 mol (36 g) Guaiacoxyethylbromide and 0.186 mol (27.3 g) phthalimide was dissolved in 100 mL dimethylacetamide, refluxed and stirred the solution under 90° C., after 30 minute, dropping in KOH-Methanol solution. The KOH-Methanol solution was dissolved 0.186 mol (10.45 g) KOH in 30 ml Methanol. The continue refluxed for 1.5 hrs until the reaction completely.

Cooled the mixture and pour into 300 mL H₂O, filtered the sold and dissolved in 200 mL 10% K₂CO₃ solution, stirred and heat for 10 minutes. Filtered the soil and wash with H₂O, recrystalized with anhydrous alcohol, to obtain the pure white compound.

(3) Prepared Guaiacoxyethylamine

0.071 mol (2 lg) N-[2-(2-chlorophenoxy)ethyl]phthalimide and 0.071 mol (3.55 g) hydrazine hydrate were dissolved in 70 mL anhydrous alcohol, refluxed and stirred the solution for 45 minute, then continue refluxed and stirring for 1 hour after add 20 mL 18% HCl solution. Cooling and concentrated, then alkalized the residuer with 20% NaOH solution, extract the mixture with CH₃Cl and through K₂CO₃ to drying. Then the solvent was removed tinder reduced pressure, the concentrator was pour into silica gel column, purified by chromatography, eluated with ethyl acetate, to obtain the compound.

### EXAMPLE 4

### Synthesis of Compound 1

### (Propionolol, 1-[(4 -Propenoic acid ethyl ester)phenoxy-3-amino]-propanol)

0.03 mol (6.6 g) 4-epoxylphenylpropionic acid ethyl ester and 0.03 mol (5.0 g) tert-butylamine were dissolved in 30 mL anhydrous alcohol, continued to reflux for 2 hours under 55° C. Then removed the solvent under reduced pressure, and pour concentrator into silica gel column, purified by chromatography, eluated with equal ratio of n-hexane and ether, then recrystalized to obtain the pure white compound.

### EXAMPLE 5

### Synthesis of Compound 2 (Vanynolol)

0.03 mol (6.6 g) 4-epoxyphenylmethyl alcohol and 0.03 mol (5.0 g) tert-butylamine were dissolved in 30 mL anhydrous alcohol, continued to stirr for 2 hours at room temperature. The solvent of mixture was removed under reduced pressure, the concentrator was pour into silica gel column, purified by chromatography, eluated with n-hexane and ethyl acetate, to obtain the compound. Recrystalized with n-hexane to obtain the pure white compound.

### EXAMPLE 6

### Synthesis of Compound 3 (Propiodilol)

03 mol (6.6 g,) 4-epoxylphenylpropionic acid ethyl ester and 0.03 mol (5.0 g) guaiacoxyethylamine were dissolved in 30 mL anhydrous alcohol, continue stirred for 2 hours at room temperature. The solvent of mixture was removed under reduced pressure, the concentrator was pour into silica gel column, purified by chromatography, eluated with n-hexane and ethyl acetate, to obtain the compound. recrystalized with n-hexane to obtain the pure white compound.

### EXAMPLE 7

### Synthesis of Compound 4 (Vanydilol )

0.03 mol (6.6 g) 4-epoxybenzyl alcohol and 0.03 mol (5.0 g) guaiacoxyethylamine were dissolved in 30 mL anhydrous alcohol continue stirred for 2 hours at room temperature. The solvent of mixture was removed under reduced pressure, the concentrator was pour into silica gel column, purified by chromatography, eluated with n-hexane and ethyl acetate, to obtain the compound, recrystalized with n-hexane to obtain the pure white compound.

### EXAMPLE 8

### Synthesis of Compound 5 (Betanapril)

Mixed 10 g compound 1(Propionolol), 1.0 g NaOEt, and 6 g (COOEt)₂, then heated to 60-70° C. for 45 minute, cooled and 200 ml distilled water was added and neutralize with conc. HCl. Divided the ethyl acetate layer at separate funnel, add 90 ml DMSO into aqua layer, then continued to reflux under 140-150° C. for 45 minute after add 10 ml aqua solution mixture of 1.5 g LiCl. Cooled the mixture and extract with ethyl acetate, to obtain the hazel aqua solution. Then the solvent was removed under reduced pressure, the concentrator was pour into silica gel column, purified by chromatography, eluated with equal ratio of Methanol and ethyl acetate, recrystalized with Methanol, to obtain the pure white compound.

The ethanol-L-alanine-proline tert-butyl ester solution formed by dissolving 2 g L-alanine-proline tert-butyl ester into ethanol Dissolved the said pure white compound in 85 ml ethanol-L-alanine-proline tert-butyl ester solution and continue stirred for 1 hrs.

Slowly dissolved 1.5 g sodium cyanoborohydride in 85 ml ethanol to form ethanol-sodium cyanoborohydride. Continue stirred over night after added this solution. Cooled the mixture and extract with ethyl acetate, dried with anhydrous NaSO₄, the solvent was removed under reduced pressure. Dissolved residuer with n-hexane, continue refluxed under 40° C. for 30 hrs after added 20 ml CH₃COOH. Then removed the solvent to obtain the pure white compound.

### EXAMPLE 9

### Another Synthesis Method of Compound 5 (Betanapril)

Mixed 10 g compound 1 (Propionolol), 1.0 g NaOEt, and 6 g (COOEt)₂, then heated to 60-70° C. for 45 minute, cooled and 200 ml distilled water was added and neutralize with conc. HCl. Divided the ethyl acetate layer at separate funnel, add 90 ml DMSO into aqua layer, then continue refluxed under 140-150° C. for 45 minute after add 10 ml aqua solution mixture of 1.5 g LiCl. Cooled the mixture and extract with ethyl acetate, to obtain the hazel aqua solution. Then the solvent was removed under reduced pressure, the concentrator was pour into silica gel column, purified by chromatography, eluated with equal ratio of Methanol and ethyl acetate, recrystalized with Methanol, to obtain the pure white compound.

The ethanol-L-alanine-proline tert-butyl ester solution formed by dissolving 2 g L-alanine-prolinie tert-butyl ester HCl into alkali ethanol. Dissolved the said pure white compound in 85 ml ethanol-L-alanine-proline tert-butyl ester solution and continue to stir for 1 hrs. Slowly dissolved 1.5 g sodium cyanoborohydride in 85 ml ethanol to form ethanol-sodium cyanoborohydride, then continue to stir over night after added with the former solution.

Cooled the mixture and extracted with ethyl acetate, then dried with anhydrous NaSO₄, the solvent was removed under reduced pressure. Dissolved residuer with n-hexane, continue refluxed under 40° C. for 30 hrs after added 20 ml CH₃COOH. Then continue refluxed for 3 hrs after added Proline benzyl ester-DEPC solution and Et₃N-DMF solution.

The said proline benzyl ester-DEPC solution was prepared as follow, dissolved Proline benzyl ester HCl and condensing agent in 120 ml alkali DMF. Whether 1.4 g diethyl phosphorocyanidate (DEPC) was selected as condensing agent. Et₃N-DMF solution was prepared as 2 g Et₃N dissolved in 20 ml DMF. Washed with mixture solution of ethyl acetate, 10% H₃PO₄, 1N NaOH, and H₂O, when the reaction completed. Then removed the ethyl acetate layer to obtain the white solid.

Dissolved the white solid with 20 ml CH₃COOH, continue to reflux under 40° C. for 30 hrs after added with 1 g diethyl phosphorocyanidate (DEPC). The concentrate was pour into silica gel column after removed the solvent, purified by chromatography, eluated with n-hexane and ethyl acetate, to obtain the compound. recrystalized with methanol to obtain the pure white compound.

### EXAMPLE 10

### Synthesis of Compound 6 (Labetapril)

Mix 10 g compound 3 (Propiodilol), 1.0 g NaOEt, and 6 g (COOEt)₂, then heated to 60-70° C. for 45 minute, cooled and 200 ml distilled water was added and neutralized with conc. HCl. Divided the ethyl acetate layer at separate funnel, add 90 ml DMSO into aqua layer, then continued reflux under 140-150° C. for 45minute after add 10 ml aqua solution mixture of 1.5 g LiCl. Cooled the mixture and extracted with ethyl acetate to obtain the hazel aqua solution. Then the solvent was removed under reduced pressure, the concentrator was pour into silica gel column, purified by chromatography, eluated with equal ratio of methanol and ethyl acetate, recrystalized with methanol to obtain the pure white compound.

The ethanol-L-alanine-proline tert-butyl ester solution formed by dissolving 2 g L-alanine-proline tert-butyl ester into alkai ethanol. Dissolved the said pure white compound in 85 ml ethanol-L-alanine-proline tert-butyl ester solution and continue to stir for 1 hrs.

Slowly dissolved 1.5 g sodium cyanoborohydride in 85 ml ethanol to form ethanol-sodium cyanoborohydride. Continued to stir over night after added with this solution. Cooled the mixture and extract with ethyl acetate, dried with anhydrous NaSO₄, the solvent was removed under reduced pressure. Dissolved residuer with n-hexane, continue to reflux under 40° C. for 30 hrs after added with 20 ml CH₃COOH. Then removed the solvent and recrystalized with methanol, to obtain the pure white compound.

### EXAMPLE 11

### Another Synthesis Method of Compound 6 (Labetapril)

Mixed 10 g compound 1 (Propionolol), 1.0 g NaOEt, and 6 g (COOEt)₂, then heated to 60-70° C. for 45 minute, cooled and 200 ml distilled water was added and neutralized with conc. HCl. Divided the ethyl acetate layer at separate funnel, add 90 ml DMSO into aqua layer, then continued to reflux under 140-150° C. for 45 minute after add 10 ml aqua solution mixture of 1.5 g LiCl. Cooled the mixture and extracted with ethyl acetate, to obtain the hazel aqua solution. Then the solvent was removed under reduced pressure, the concentrate was pour into silica gel column, purified by chromatography, eluated with equal ratio of methanol and ethyl acetate, recrystalized with methanol, to obtain the pure white compound.

The ethanol-L-alanine-proline tert-butyl ester solution was formed by dissolving 2 g L-alanine-proline tert-butyl ester HCl into alkai ethanol. Dissolved the said pure white compound in 85 ml ethanol-L-alanine-proline tert-butyl ester solution and continue stirred for 1 hrs. Slowly dissolved 1.5 g sodium cyanoborohydride in 85 ml ethanol to form ethanol-sodium cyanoborohydride. Continue to stirr over night after added with this solution.

Cooled the mixture and extract with ethyl acetate, dried with anhydrous NaSO₄, the solvent was removed under reduced pressure. Dissolved residuer with n-hexane, continue to reflux under 40° C. for 30 hrs after added 20 ml CH₃COOH. Then continue to reflux for 3 hrs after added with Proline benzyl ester-DEPC solution and Et₃N-DMF solution.

The said proline benzyl ester-DEPC solution was prepared as follow, dissolved proline benzyl ester HCl and condensing agent in 120 ml DMF. Whether 1.4 g diethyl phosphorocyanidate (DEPC) was selected as condensing agent. Et₃N-DMF solution was prepared as 2 g Et₃N dissolved in 20 ml DMF. Washed with mixture solution of ethyl acetate, 10% H₃PO₄, 1N NaOH, and H₂O, when the reaction completed. Then removed the ethyl acetate layer to obtain the white solid.

Dissolved the white sold with 20 ml CH₃COOH, continue to reflux under 40° C. for 30 hrs after added with 1 g diethyl phosphorocyanidate (DEPC). The concentrate was pour into silica gel column after removed the solvent, purified by chromatography, eluated with n-hexane and ethyl acetate, to obtain the compound, recrystalized with methanol to obtain the pure white compound.

### EXAMPLE 12

### Synthesis of Compound 7 (Vanynolpril)

Dissolved 4 g enalapril in 30 ml Tetrahydrofuran, refluxed and stirred the solution for 3 hrs after add 3.5 g compound 2 Vanynolol, and 2 ml SOCl₂. Then the solvent was removed under reduced pressure, the concentrator was pour into silica gel column, purified by chromatography, eluated with equal ratio of n-hexane and ethyl acetate, to obtain the pure white crystal compound. Recrystalized with methanol to obtain the pure 4.9 g white compound.

### EXAMPLE 13

### Synthesis of Compound 8 (Vanydilopril)

Dissolved 4 g enalapril in 30 ml Tetrahydrofuran, refluxed and stirred the solution for 3 hrs after add 3.5 g compound 2 Vanynolol, and 2 ml SOCl₂. Then the solvent was removed under reduced pressure, the concentrator was pour into silica gel column, purified by chromatography, eluated with equal ratio of n-hexane and ethyl acetate, to obtain the pure white crystal compound. Recrystalized with methanol to obtain the pure 4.1 g white compound.

TABLE 1
4-epoxylphenylpropionic acid ethyl ester

| | | |
|---|---|---|
| MS | 249 | m.p. |
| (Scan FAB⁺) | | |

| IR | 3300 cm⁻¹(—NH— group), | |
|---|---|---|
| | 1700 cm⁻¹(carbonyl , —COOR group) | |
| ¹H-NMR | 1.34(t, 3H, —COOCH₂CH₃) | 2.58-2.64(m, 4H, Ar—CH₂H₂CO) |
| (CDCl₃) | 2.76(q, J = 7.14, 1H, —CHCH₂—) | 2.90(t, J = 4.8, 1H, CH—CH₂) |
| | 3.34-3.42(m, 1H, CH—CH₂) | 4.1-4.3(m, 2H, ArOCH₂—) |
| | 4.20(q, J = 7.14, 2H, —COOCH₂CH₃) | 6.86-7.04(m, 7H, Ar—H) |

4-epoxylbenzylalcohol

| | | |
|---|---|---|
| MS | 249 | m.p. |
| (Scan FAB⁺) | | |

| IR | 3300 cm⁻¹(—NH— group), | |
|---|---|---|
| | 3200 cm⁻¹(—OH— group) | |
| ¹H-NMR | δ 2.76(q, J = 7.14, 1H, —CHCH₂—) | 2.90(t, J = 4.8, 1H, CH—CH₂) |
| (CDCl₃) | 3.34-3.42(m, 1H, CH—CH₂) | 4.1-4.3(m, 2H, ArOCH₂—) |
| | 3.85(s, 3H, ArOCH₃) | 4.61(s, 2H, Ar—CH₂OH) |
| | 6.86-7.04(m, 7H, Ar—H) | |

compound 1 (Propionolol)

| Formula(MW) | C₁₈H₂₉NO₄(323) | | |
|---|---|---|---|
| Yield % | 92 | | |
| MS | | m.p. | 185° C. |
| (Scan FAB⁺) | | | |

| IR | 3300 cm⁻¹(—NH— group) | |
|---|---|---|
| ¹H-NMR | δ 1.12(s, 9H, CH₃x3) | 1.6(s, 2H, CH₂) |
| (CDCl₃) | 1.8-2.2(m, 2H, CH₂) | 2.4-2.61(m, 4H, 2xAr—CH₂O) |
| | 2.5-2.8(m, 2H, —CH₂—) | 2.93-3.18(m, 4H, —CH₂NHCH₂—) |
| | 3.85(s, 3H, ArOCH₃) | 4.06-4.14(m, 2H, ArOCH₂—) |
| | 4.17-4.20(m, 2H, —COOCH₂CH₃) | |
| | 4.24-4.31(m, 1H, CH(OH)) | 6.86-7.04(m, 12H, Ar—H) |

compound 2 (Vanynolol)

| Formula(MW) | C₂₃H₃₁NO₆(417) | | |
|---|---|---|---|
| Yield(%) | 85 | | |
| MS | | m.p. | 173° C. |
| (Scan FAB⁺) | | | |

| IR | 3300 cm⁻¹(—NH— group) | |
|---|---|---|
| ¹H-NMR | δ 1.12(s, 9H, CH₃x3) | 2.61(s, 2H, Ar—CH₂O) |
| (CDCl₃) | 2.93-3.18(m, 4H, —CH₂NHCH₂—) | 3.85(s, 3H, ArOCH₃) |
| | 4.06-4.14(m, 2H, ArOCH₂—) | 4.24-4.31(m, 1H, CH(OH)) |
| | 6.86-7.04(m, 7H, Ar—H) | |

compound 3 (Propiodilol)

| Formula(MW) | C₁₅NO₄H₂₅(283) | | |
|---|---|---|---|
| Yield(%) | 89 | | |
| MS | 433 | m.p. | 168° C. |
| (Scan FAB⁺) | | | |

| IR | IR: 3300 cm⁻¹ (—NH— group) 3200 cm⁻¹ (—OH— group) | |
|---|---|---|
| | 1700 cm⁻¹ (carbonyl, —COOR group) | |
| ¹H-NMR | δ 1.34(t, 3H, —COOCH₂CH₃) | 2.58-2.64(m, 4H, Ar—CH₂H₂CO) |
| (CDCl₃) | 2.93-3.18(m, 4H, —CH₂NHCH₂—) | 4.06-4.14(m, 2H, ArOCH₂—) |
| | 4.17-4.20(m, 2H, —COOCH₂CH₃) | 4.24-4.31(m, 1H, CH(OH)) |
| | 6.86-7.04(m, 7H, Ar—H) | |

compound 4 (Vanydilol)

| Formula (MW) | C₂₀NO₆H₂₇(377) | | |
|---|---|---|---|
| Yield(%) | 74 | | |
| MS | 377 | m.p. | 158° C. |
| (Scan FAB⁺) | | | |

| IR | 3300 cm⁻¹(—NH— group) | |
|---|---|---|
| ¹H-NMR | δ 2.93-3.18(m, 4H, —CH₂NHCH₂—) | 3.77-3.85(s, 3H, ArOCH₃) |
| (CDCl₃) | 4.06-4.14(m, 2H, ArOCH₂—) | 4.24-4.31(m, 1H, CH(OH) |
| | 4.61(Ar—CH₂O) | 6.86-7.04(m, 7H, Ar—H) |

compound 5 (Betanapril)

| Formula MW | C₂₇H₄₄N₃O₇(522) | | |
|---|---|---|---|
| Yield % | 68 | | |
| MS | 522 | m.p. | 168° C. |
| (Scan FAB⁺) | | | |

| IR | 3300 cm⁻¹(—NH— group) | |
|---|---|---|
| | 4.17-4.20(m, 2H, —COOCH₂CH₃) | |
| ¹H-NMR | 1.2-1.4(m, 12H, CH₃x4) | 1.6(s, 2H, CH₂) |
| (CDCl₃) | 1.8-2.2(m, 2H, CH₂) | 2.4-2.61(m, 4H, 2xAr—CH₂O |
| | 2.5-2.8(m, 2H, —CH₂—) | 2.93-3.18(m, 4H, —CH₂NHCH₂—) |
| | 3.85(s, 3H, ArOCH₃) | 4.06-4.14(m, 2H, ArOCH₂—) |
| | 4.17-4.20(m, 2H, —COOCH₂CH₃) | |
| | 4.24-4.31(m, 1H, CH(OH)) | 6.86-7.04(m, 4H, Ar—H) |

compound 6 (Labetapril)

| Formula(MW) | C₃₂H₄₆N₃O₉(616) | | |
|---|---|---|---|
| Yield(%) | 66 | | |
| MS | 433 | m.p. | 164° C. |
| (Scan FAB⁺) | | | |

| IR | 3300 cm⁻¹(—NH— group) 3200 cm⁻¹ (—OH— group) | |
|---|---|---|
| | 1700 cm⁻¹(carbonyl, —COOR group) | |
| ¹H-NMR | δ 1.1(s, 3H, CH₃) | 1.34(t, 3H, —COOCH₂CH₃) |
| (CDCl₃) | 1.9(s, 1H, CH) | 2.58-2.64(m, 4H, Ar—CH₂H₂CO) |
| | 2.93-3.18(m, 4H, —CH₂NHCH₂—) | 4.06-4.14(m, 2H, ArOCH₂—) |
| | 4.17-4.20(m, 2H, —COOCH₂CH₃) | 4.24-4.31(m, 1H, CH(OH)) |
| | 6.86-7.04(m, 7H, Ar—H) | |

compound 7 (Vanynolpril)

| Formula(MW) | C₃₅N₃O₈H₅₁(641) | | |
|---|---|---|---|
| Yield(%) | 58 | | |
| MS | 641 | m.p. | 152° C. |
| (Scan FAB⁺) | | | |

| IR | 3300 cm⁻¹(—NH— group) | |
|---|---|---|
| ¹H-NMR | δ 1.12(s, 9H, CH₃x3) | 1.6(s, 2H, CH₂) |
| (CDCl₃) | 1.8-2.2(m, 2H, CH₂) | 2.4-2.61(m, 4H, 2xAr—CH₂O) |
| | 2.5-2.8(m, 2H, —CH₂—) | 2.93-3.18(m, 4H, —CH₂NHCH₂—) |
| | 3.85(s, 3H, ArOCH₃) | 4.06-4.14(m, 2H, ArOCH₂—) |
| | 4.17-4.20(m, 2H, —COOCH₂CH₃) | |
| | 4.24-4.31(m, 1H, CH(OH)) | 6.86-7.04(m, 12H, Ar—H) |

compound 8 (Vanydilopril)

| Formula(MW) | C₄₀N₃O₁₀H₅₃(735) | | |
|---|---|---|---|
| Yield(%) | 53 | | |
| MS | 735 | m.p. | 156° C. |
| (Scan FAB⁺) | | | |

| IR | 3300 cm⁻¹(—NH— group) | |
|---|---|---|
| ¹H-NMR | δ 1.6(s, 2H, CH₂) | 1.8-2.2(m, 2H, CH₂) |
| (CDCl₃) | 2.4-2.61(m, 4H, 2xAr—CH₂O) | 2.5-2.8(m, 2H, —CH₂—) |
| | 2.93-3.18(m, 4H, —CH₂NHCH₂—) | 3.85(s, 3H, ArOCH₃) |
| | 4.06-4.14(m, 2H, ArOCH₂—) | |
| | 4.17-4.20(m, 2H, —COOCH₂CH₃) | 4.24-4.31(m, 1H, CH(OH)) |
| | 6.86-7.04(m, 12H, Ar—H) | |

TABLE 2

| compound (2 mg.kg, i.v) | Decrease of blood pressure (mmHg) |
|---|---|
| compound 1 | 60 ± 4.7 |
| compound 2 | 72 ± 2.5 |
| compound 3 | 56 ± 3.8 |
| compound 4 | 83 ± 9.1 |
| compound 5 | 52 ± 4.8 |
| compound 6 | 65 ± 9.2 |
| compound 7 | 55 ± 6.3 |
| compound 8 | 63 ± 7.2 |

TABLE 3
pA₂ and β₁/β₂-Selectivity Values from In Vitro Experiments

| | β₁ | | | | |
|---|---|---|---|---|---|
| | pA₂ value^{a} | | β₂ | α₁ | |
| | Right | Left | PA₂ value^{a} | pA₂ value^{a} | β₁/β₂ |
| compounds | atrium | atrium | Trachea | Aorta | Ratio |
| 1 | 6.83 | 6.92 | 6.43 | | 2.1 |
| 2 | 6.67 | 6.84 | 6.61 | 7.23 | 2.3 |
| 3 | 6.74 | 6.78 | 6.71 | | 1.9 |
| 4 | 6.72 | 6.76 | 6.69 | 7.89 | 1.6 |
| 5 | 5.92 | 5.84 | 5.79 | | 1.7 |
| 6 | 5.41 | 5.31 | 5.43 | 6.93 | 1.4 |
| 7 | 5.87 | 5.78 | 5.64 | | 1.9 |
| 8 | 5.28 | 5.19 | 5.11 | 6.85 | 2.2 |
| propranolol | 8.24 ± | 8.07 ± | 8.07 ± | <5.00 | 1.5 |
| | 0.06 | 0.12 | 0.12 | | |
| labetalol | 7.91 ± | 7.54 ± | 7.54 ± | 6.87 ± | 2.3 |
| | 0.09 | 0.16 | 0.16 | 0.08 | |
| atenolol | 7.34 ± | 5.70 ± | 5.81 ± | <5.00 | 34.7 |
| | 0.03 | 0.06 | 0.06 | | |

^{a}pA₂ values were obtained from the formula pA₂ = [log(DR− 1)− log molar concentration antagonist] and calculated from individual Schild plot by regression. Each value was the mean ± SEM of six to eight experimental results.
^{b}The β₁/β₂-selectivity ratios were obtained from the antilogarithm of the differences between the mean pA₂ values obtained from right atrium and trachea.

TABLE 4
Inhibitory activity of angiotensin converting enzyme(ACE) by
various ACE inhibitors

| | In Rat Serum (%) | | | In Rat lung (%) | | |
|---|---|---|---|---|---|---|
| compounds | 10⁻⁶M | 10⁻⁷M | 10⁻⁸M | 10⁻⁶M | 10⁻⁷M | 10^{31 8}M |
| Enalapril | 99 | 94 | 54 | 98 | 93 | 51 |
| compound 5 | 86 | 72 | 32 | 89 | 76 | 38 |
| compound 6 | 81 | 67 | 28 | 84 | 72 | 33 |
| compound 7 | 83 | 74 | 35 | 81 | 68 | 31 |
| compound 8 | 79 | 68 | 29 | 78 | 63 | 29 |

Each data represent mean value of three samples

What is claimed is

1. The phenoxypropanolamine derivative compound of formula I, 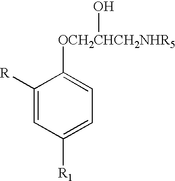
whether
R is selected from the groups of hydrogen, unsaturated 2-6 straight chain carbon atoms, saturated 1-6 straight chain carbon atoms, unsaturated 2-6 straight chain of alkoxyl group, saturated 1-6 straight chain of alkoxyl group, halogen, and —NO₂;
R₁ is selected from the groups of —R₂OH, —R₂OR₃, —R₂COCOOR₄, —R₂COOR₄, R₂CH(COR₄)-alanylproline, and R2CH(COOR4)-alanylproline;
R₂ is selected from the groups of unsaturated 2-6 straight chain carbon atoms, saturated 1-6 straight chain carbon atoms;
R₃ is selected from the ester groups of peptides which were, on ACEI (Angiotensin Converting Enzyme Inhibitor) terminal structure;
R₄ is selected from the groups of hydrogen, unsaturated 2-6 straight chain carbon atoms, saturated 1-6 straight chain carbon atoms;
R₅ is selected from the groups of hydrogen, unsaturated 2-6 straight chain carbon atoms, saturated 1-6 straight chain carbon atoms, and 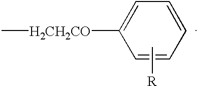
R may be on the meta, ortho, or para position with ethoxyl group on the bezene ring.

2. A pharmaceutical compound which has α/β-adrenoceptors and β-adrenoceptor binding affinity, and using formula I as the main component, various diluents and excipients could be included when necessary.

3. A pharmaceutical compound which has Angiotensin Converting Enzyme Inhibitivity (ACEI), and using formula I as the main component, various diluents and excipients could be included when necessary.

4. The prepared methods of formula I derivatives compounds, Added 4-hydroxylphenylpropionic acid and ethanol into reaction bottle, reaction was carried out with NaOH and epichlorohydrin; till the reaction was complete, then removed the solvent, purified by chromatography, to obtain the pure white crystal compound 4-epoxylphenylpropionic acid ethyl ester; continued to reflux 4-epoxylphenylpropionic acid ethyl ester with guaiacoxyethylamine in anhydrous alcohol; then removed the solvent and purified by chromatography, eluated with equal ratio of n-hexane and ether, then recrystalized to obtain the pure compound 3 (Propiodilol);
Mixed compound 3 (Propiodilol), NaOEt, and (COOEt)₂, then heated to 60-70° C., cooled and distilled water was added and neutralized with conc. HCl; divided the ethyl acetate layer at separate funnel, add DMSO into aqua layer, then continue refluxed after add aqua solution mixture of LiCl; cooled the mixture and extract with ethyl acetate, then purified by chromatography, eluated with equal ratio of methanol and ethyl acetate, recrystalized with Methanol, to obtain the pure white compound;
Dissolved the pure white compound in ethanol-L-alanine-proline tert-butyl ester solution and continued to stirr over night after added ethanol-sodium cyanoborohydride; cooled the mixture and extract with ethyl acetate, dried with anhydrous NaSO₄, the solvent was removed under reduced pressure. Dissolved residuer with n-hexane, continue refluxed after added CH₃COOH; then removed the solvent and recrystalized with methanol, to obtain the compound 6 (Labetapril).

5. The preparation of formula I derivatives were obtained by heating compound 1 (Propionolol), NaOEt, and (COOEt)₂, at 45° C., then cooled and distilled water was added and neutralize with conc. HCl; divided the ethyl acetate layer at separate funnel, added DMSO into aqua layer, then continued to reflux after added aqua solution mixture of LiCl; cooled the mixture and extract with ethyl acetate, to obtain the hazel aqua solution; then purified by chromatography, eluated with equal ratio of methanol and ethyl acetate, recrystalized with methanol, to obtain the pure white compound;
Dissolved the pure white compound in ethanol-L-alanine-proline tert-butyl ester solution and continue stirre over night after added ethanol-sodium cyanoborohydride. Cooled the mixture and extract with ethyl acetate, dried with anhydrous NaSO₄, the solvent was removed under reduced pressure; dissolved residuer with n-hexane, continue refluxed under after added CH₃COOH, Proline benzyl ester-DEPC solution and Et₃N-DMF solution. Wash with mixture solution of ethyl acetate, 10% H₃PO₄, 1N NaOH, and H₂O, when the reaction completed; then removed the ethyl acetate layer to obtain the white sold;
dissolved the white sold with CH₃COOH, continue refluxed added diethyl phosphorocyanidate (DEPC); the concentrator was purified by chromatography, eluated with n-hexane and ethyl acetate, to obtain the compound. recrystalized with Methanol, to obtain the compound 6 (Labetapril).
